(19) United States
(12) Patent Application Publication
(10) Pub. No.: US 2007/0010480 A1
(43) Pub. Date: Jan. 11, 2007

(54) Dietary food items for a weight control or weight loss diet

(75) Inventors: Matthias Rusing, Koln (DE); Stephan Hausmanns, Wiesbaden (DE)

Correspondence Address: MERCHANT & GOULD PC, P.O. BOX 2903, MINNEAPOLIS, MN, 55402-0903, US

(73) Assignee: NUTRINOVA Nutrition Specialties & Food Ingredients GmbH, Frankfurt am Main (DE)

(21) Appl. No.: 10/558,320

(22) PCT Filed: May 26, 2004
(86) PCT No.: PCT/EP04/05666

§ 371 Date: Nov. 23, 2005

(30) Foreign Application Priority Data
May 28, 2003 (DE) 103 24 548.0

(51) Int. Cl. A61K 31/718; A61K 31/716; A61K 31/202; A61K 31/715

(52) U.S. Cl. 514/54; 514/60; 514/560

(57) ABSTRACT

The invention relates to dietetic foods in the context of weight control or weight reduction, comprising at least one n3 fatty acid (omega-3) in combination with dietary fibers. Advantageously, the foods further comprise a balanced ratio of carbohydrates, protein and fat. Preferably, the inventive foods comprise a balanced ratio of fatty acids, in particular relating to the ratio n3 to n6 fatty acids (approximately 1:1 to 1:10). The inventive food combinations serve the purpose of a sufficient supply of essential fatty acids, in particular long-chain omega-3 fatty acids, in particular docosahexaenoic acid (DHA), in the context of a targeted weight reduction. The inventive food combinations are of particular advantage in the case of restricted fat supply, e.g. in the context of a reduced-fat diet with the purpose of weight reduction or weight control. In addition, the invention relates to a method for producing such dietetic foods, and also to their use.

Overweight and obesity (adiposity) is a social disease and is currently understood as a chronic health disorder. Overweight, in the rich industrial countries, is the most widespread diet-related (and thus preventable) risk factor. The World Health Organization (WHO) in the meantime is already speaking of an “adiposity epidemic”.

It is known that overweight occurs when the daily calorie intake in the long term exceeds the energy consumption of the body (positive energy balance). Overweight can lead to a multiplicity of complications which relate to virtually all organ systems of the body and can lead to an increased risk of a great number of serious chronic disorders and a reduced life expectation:

coronary heart disorders, heart attack

heart enlargement with accompanying heart weakness,

elevated blood pressure (hypertension)

elevated blood fats (cholesterol and triglycerides)

arteriosclerosis

stroke

sleep apnea syndrome

diabetes type 2

gout

gallstones (cholecystolithiasis)

joint damage (arthroses) to spinal column, hip and knee joints

pregnancy complications

For these reasons, prevention of overweight, and in particular adiposity, or a weight-reducing diet in the case of overweight which is already present is extraordinarily important.

In the prior art, a distinction is made between various diets for reduction of overweight:

The reduced-calorie mixed diet should lead to an energy deficit of at least 500 kcal/day. In the diet, 50-55% of the energy is to be available from carbohydrates, 30% from fat and 15-20% from protein (at least 50 g of high biological value protein) (Deutsche Gesellschaft für Ernährung (DGE) [German nutrition society] 1991, Empfehlungen für die Nährstoffzufuhr. [Recommendations for nutrient supply] 5th revision. Umschau-Verlag, Frankfurt, 1991).

Reduction diets having an energy content of 700-1000 kcal/day shall, in accordance with section 14a of the German diet ordinance, contain 50 g of protein, 90 g of complex carbohydrates and 7 g of linoleic acid (German diet ordinance 1988). According to directive 89/398 of the EC Commission of Feb. 26, 1996, on foods for a low-calorie diet for weight reduction, the daily ration shall contain at least 800 kcal. At least 25%, but at most 50%, of the calorific value can be assigned to proteins. The calorific value of the fats shall not exceed 30% of the total calorific value. At least 4.5 g of linoleic acid shall be present. The daily ration shall contain at least 10 g and at most 30 g of dietary fiber per daily ration. The amount of carbohydrate arises as supplementation to the fats and proteins in the permitted energy range.

Very low calorie diets (VLCD) having an energy content of 450-700 kcal/day shall contain at least 50 g of protein, 45 g of carbohydrates and 7 g of fat with simultaneous substitution of minerals, vitamins and trace elements for the isocaloric requirements. They may be used only on prescription and should not be used for more than four to six weeks and remain reserved for high-risk patients where, for urgent medical reasons, rapid weight reduction is necessary (Anderson et al., 1992, Benefits and risks of an intensive very-low-calorie diet program for severe obesity, Am. J. Gastroenterol. 87, 6-15).

However, in particular in the case of overweight which is already manifest, weight reduction is achieved by reduction in the daily fat supply, which can be the cause for a great number of serious problems.

This is because many of the currently propagated diets for weight reduction, in particular many of the so-called “crash diets” are accompanied with significant health risks.

After a relatively large weight decrease by, for example, reduction of the fat content of the diet, frequently directly after ending the diet, a massive weight increase recurs, that is termed the yo-yo effect. A cyclic change in body weight of this type is, however, despite the high risks which accompany overweight, frequently more harmful to health than keeping the possibly excess weight stable for a long period. For instance, the mortality, in particular in connection with coronary heart disorders, in individuals having cyclically varying body weight is significantly higher than in individuals having a constant weight. Furthermore, extremely adverse effects of yo-yo diets on the psyche have been demonstrated, for example (see for example Brownell, K. D., et al., 1994, Medical, metabolic, and psychological effects of weight cycling, Arch. Intern. Med. 154, 1325-30).

It is known that many different organizations (WHO, FAO, AHA; ISSFAL, British Nutrition Foundation and many others, see e.g. Krauss et al. AHA Dietary Guidelines. Revision 2000: A statement for healthcare professionals from the nutrition committee of the American Heart Association. Circ. 2000, 2296-2311) advise significantly increasing the supply of n3 fatty acids. Most recommendations here relate to the supply of n3 fatty acids (in particular DHA and EPA) by the regular consumption (at least 2× per week) of marine fish, in particular those having a high fat content.

Furthermore, various scientific studies are known which describe generally advantageous compositions of diet ingredients for weight reduction and/or cardiovascular health (Gohlke, Z Kardiol 2002;91 Suppl 2:12-24; Platt, Current concepts in optimum nutrition for cardiovascular disease, Am J Cardiol Jan. 15, 2002;89(2):229-31).

Various preparations containing fatty acid(s) or fatty acid mixtures have been developed for the diet or for food supplementation with particular nutritional objectives.

For instance, U.S. Pat. No. 4,526,902 describes a preparation containing n3 fatty acids for the treatment and prophylaxis of thromboembolic states. European patent EP 0 275 643 B1 relates to a composition containing linoleic acid, inter alia for improving the memory.

In addition, e.g. in the Laid-Open application DE 39 24 607 A1, fatty acid mixtures of omega-3 and omega-6 fatty acids are described which have a beneficial action on cardiovascular risk factors.

European patent application EP 0 756 827 A2 describes fatty acid mixtures in combination with other food components for the diet of e.g. patients having weakened immune function or tumor patients.

However, the abovedescribed prior art is disadvantageous in that, within the individual diet components, there has been insufficient differentiation between various substances within a group of food components (e.g. dietary fibers, fatty acids, proteins).

For example, within the group of the omega-3 fatty acids, it is necessary to make a clear distinction between ALA, EPA and DHA in their physiological importance. The same applies to the various components from the large group of dietary fibers which can be greatly different in their physiological importance (soluble, insoluble, prebiotic dietary fibers etc.). Certain dietary fibers can, for example, lead to the fatty acids supplied not being physiologically available. These circumstances can lead to the diet being interrupted, or after termination of, or during, the diet, there being a deficit of essential components, which can possibly lead to an increased feeling of hunger or corresponding deficiency symptoms. Furthermore, an increased health risk due to a deficiency of essential components can exist, e.g. in the field of cardiovascular disorders.

Considering the prior art, it was therefore an object of the present invention to provide dietetic preparations for use in the context of health-promoting weight reduction, in particular in the case of overweight or adiposity, or a health-maintaining weight-controlling diet.

In particular, the preparation should make it possible to enable a relatively long-term change of diet and lead to an increase in wellbeing without having said disadvantages of the reduced-fat and reduced-calorie dietetic compositions for weight reduction previously known in the prior art. The dietetic preparation should advantageously be useful in the prevention of the yo-yo effect, and counteract undersupply with essential fatty acids.

These and other objects not explicitly mentioned, but which are able to be derived or concluded without problems from the associations discussed herein at the outset are solved by the subject matter defined in the claims of the present invention.

An advantageous dietetic preparation is provided by the preparation defined in claim 1. The inventive dietetic preparation comprises at least one n3 fatty acid and also if appropriate at least one dietary fiber.

Particularly preferably, the inventive dietetic preparation further comprises if appropriate carbohydrates, proteins and/or fats.

Under a further preferred embodiment, the inventive dietetic preparation comprises
a) at least one n3 fatty acid and also if appropriate at least one n6 fatty acid, the ratio of n3 fatty acids to n6 fatty acids ranging from 1:1 to 1:10, and
   the at least one n6 fatty acid preferably being selected from the group consisting of linoleic acid, γ-linolenic acid and/or arachidonic acid, and the at least one n3 fatty acid from the group consisting of docosahexaenoic acid, eicosapentaenoic acid and/or α-linolenic acid,
b) if appropriate carbohydrates,
c) if appropriate protein,
d) if appropriate fat,
e) at least one active compound which reduces the uptake of fat in the intestine, the total energy content of the composition if appropriate being 40-75% covered, preferably 55-60%, by component b), 5-35% covered, preferably 10-15%, by component c) and 15-35% covered, preferably 25-30%, by component d), and also
f) if appropriate at least one dietary fiber.

The inventive dietetic preparation is suitable particularly for use in the context of a health-promoting weight reduction in the event of overweight or else adiposity, that is as a component of a reduced-calorie mixed diet, in a reduction diet for low-calorie diet for weight reduction or else in a very low calorie diet (VLCD).

Very particularly, however, the inventive preparation is suitable as component or supplement in a health-maintaining or promoting weight-controlling diet. Surprisingly, the inventive preparation assists in a long-term change of diet. As food component or supplement, the inventive preparation leads in the relatively long term to an increase in well being and aids in no longer leaving the weight control path once taken, which makes an occurrence of the so-called yo-yo effect less probable.

In addition, the inventive dietetic preparation helps to make up for a depletion of the body in essential fatty acids and it leads to an extremely beneficial effect on the principal risk factors for health which are due to overweight. The at least one n3 fatty acid reduces the cardiovascular risk, and also atheriosclerosis, high blood pressure, plasma triglyceride level, heart frequency variability inter alia significantly, while the at least one dietary fiber has an additional beneficial effect on the triglyceride values and in particular the cholesterol values. With regard to these lipids, dietary fibers act like a sponge. Cholesterol and triglycerides are bound, as a result of which the body cannot utilize these and they are excreted via the intestine. Precisely this effect of the dietary fibers, however, leads to the fact that the corresponding fatty acids have customarily not been combined with dietary fibers. The inventive combination, however, first makes it possible to have beneficial effects on all principal risk factors of overweight at once, and to achieve significant health advantages for people interested in weight control or reduction.

Independently of the previously described beneficial effects on cardiovascular health, the inventive dietetic preparations or products produced therefrom achieve an additional beneficial effect on health due to the increased supply of n3 fatty acids. A particular role here is played by DHA which is one of the principal components of the human brain and the retina of the eye (Horrocks, L. A. et al., 1999, Health benefits of docosahexaenoic acid., Pharmacol. Res. 40, 211-225). The use of the inventive dietetic foods also has a health-promoting effect beyond the actual nutrition in the context of a diet plan.

Dietetic preparation according to the invention is taken to mean a preparation which corresponds to the teaching of dietetics. In particular, such a preparation comprises only components which are permitted for use as foods or in pharmaceutical preparations. Furthermore, such a preparation, in particular, comprises no impurities harmful to human health.

According to the invention, the term “dietetic preparation” is taken to mean a synonym for “food, food product, preparation for parenteral nutrition”.

n3 fatty acids within the meaning of the invention are taken to mean polyunsaturated long-chain fatty acids (PUFAs) having a chain length>12 carbon atoms having at least two or more double bonds, the first of the at least two or more double bonds being constituted between carbon atoms C3 and C4 starting from the alkyl end. The n3 fatty acids can according to the invention be present not only as free fatty acids, esters, triglycerides, phospholipids, but also as other derivatives, or can have been reacted and enriched by chemical or biocatalytic transesterification, e.g. using suitable enzymes (lipases). All these substances are summarized hereinafter under the terms n3 fatty acid or n3 active compounds, which terms are used synonymously.

Inventively preferred n3 fatty acids are found in the table hereinafter:

| | | Trivial name, |
|---|---|---|
| | IUPAC-Name | abbreviation |
| C18:3 | All-cis-9,12,15- | α-Linolenic acid, |
| | Octadecatrienoic acid | ALA |
| C18:4 | All-cis-6,9,12,15- | Stearidonic acid |
| | Octadecatetraenoic acid | |
| C20:3 | All-cis-11,14,17- | |
| | Eicosatrienoic acid | |
| C20:4 | All-cis-8,11,14,17- | ETA |
| | Eicosatetraenoic acid | |
| C20:5 | All-cis-5,8,11,14,17- | Timnodonic acid, |
| | Eicosapentaenoic acid | EPA |
| C22:3 | All-cis-13,16,19- | |
| | Docosatrienoic acid | |
| C22:5 | All-cis-7,10,13,16,19- | DPA |
| | Docosapentaenoic acid | |
| C22:6 | All cis-4,7,10,13,16,19- | DHA |
| | Docosahexaenoic acid | |

Particular preference is given to docosahexaenoic acid, eicosapentaenoic acid or α-linolenic acid, DHA being very particularly preferred.

The term “dietary fibers” designates according to the invention food components which cannot be broken down by the human digestive system. Preferably, the dietary fibers and the n3 fatty acids are present in a suitable combination, which do not decrease the physiological availability of the n3 fatty acids by the sponge effect of the dietary fibers, or this effect is compensated for by a particularly high concentration of n3 fatty acids.

Inventively preferred dietary fibers are therefore indigestible carbohydrates or derivatives thereof which do not lead to a reduction of the physiological availability of the essential fatty acids.

Preference in the inventive preparation is therefore given to the use of resistant starch, fructooligosaccharides, oligofructoses and inulin or their mixture. Preference is given to a mixture of resistant starch and inulin in the ratio 1:10 to 10:1. Particular preference is given to resistant starch of type 3. Type 3 resistant starches are taken to mean an indigestible retrograded or crystalline starch (see, e.g., Cummings et al.; British Journal of Nutrition, 75 (1996), pages 733 ff.).

The inventively preferred concentration of the dietary fibers varies owing to the physiological activity of the preferred components in a range of 1-15 g per day, in particular 2-10 g per day, in a healthy adult.

A further embodiment of the present invention relates to a dietetic preparation which comprises more than one n3 fatty acid and/or more than one dietary fiber.

Preferably the dietetic preparation comprises carbohydrates, proteins and fat in an optimal combination. Particularly preferably, the combination corresponds to the recommendation of the German nutrition society, i.e. the energy fraction should consist of 40-75%, preferably 55-60%, of carbohydrates, of 5-35%, preferably 10-15%, of protein and a maximum of 15-35%, preferably 25-30%, of fat.

In a further embodiment of the present invention, the dietetic preparation additionally comprises vitamins, minerals, trace elements, further essential fatty acids and/or secondary plant materials, preferably in accordance with EC Directive 89/398 on foods for low-calorie diet for weight reduction.

A further embodiment of the present invention relates to a dietetic preparation which additionally comprises one or more n6 fatty acids.

n6 fatty acids within the meaning of the invention are taken to mean polyunsaturated long-chain fatty acids having a chain length>C12 having at least two or more double bonds, the first of the at least two or more double bonds being constituted between carbon atoms C6 arid C7 starting from the alkyl end. The n6 fatty acids can according to the invention be present not only as free fatty acids, esters, triglycerides, phospholipids but also as other derivatives, or can have been reacted and enriched by chemical or biocatalytic transesterification, e.g. using suitable enzymes (lipases). All these substances are summarized hereinafter under the term n6 fatty acid.

Inventively preferred n6 fatty acids are linoleic acid, y-linolenic acid, arachidonic acid and/or n6-DPA, linoleic acid being particularly preferred.

According to the invention, particularly preferably the ratio of n3 to n6 fatty acids in the preparation or the end product made therefrom is between 1:1 and 1:15, preferably between 1:1 and 1:10, and very particularly preferably between 1:1 and 1:5.

A further embodiment of the present invention relates to a dietetic preparation which additionally comprises one or more monounsaturated fatty acids.

An inventively preferred monounsaturated fatty acid is oleic acid.

The total fat content of the dietetic preparation or the end product made therefrom according to the invention contributes at most 35%, preferably at most 30%, to the energy which this preparation or the product provides to an organism.

The saturated fatty acid content of the dietetic preparation or of the end product made therefrom according to the invention contributes at most 10%, preferably at most 8%, of the energy which this preparation or the product contributes to an organism.

An inventively preferred dietetic preparation comprises linoleic acid, α-linolenic acid, docosahexaenoic acid and/or eicosapentaenoic acid, linoleic acid contributing 1 to 10% of the energy, preferably 2 to 3% of the energy, which this preparation or the product produced therefrom provides to the organism, α-linolenic acid 0.2-2%, preferably approximately 1%, docosahexaenoic acid and eicosapentaenoic acid together making up 0.1-1%; preferably approximately 0.3%, DHA making up at least 0.1%. In a further form, the dietetic product comprises the monounsaturated oleic acid in an amount which supplies 1-10% of the total energy. In a preferred form, the product, in addition to linoleic acid, also comprises approximately 0.1 % GLA as further n6 source.

Particular preference is given to a dietetic preparation, the docosahexaenoic content of which makes up 50-1000 mg, preferably 75-500 mg, in particular 100-300 mg, per day.

Preferably, the inventive dietetic preparations comprise one or more natural oils selected from the list: algal oil, borage oil, thistle oil, fish oil, hemp oil, linseed oil, maize germ oil, evening primrose oil, olive oil, rapeseed oil, soybean oil, nut oils and/or sunflower oil. The oils can be used in native or processed form or else serve only as source of individual fatty acids in the forms known to those skilled in the art.

In particular, linseed, or linseed oil, hemp oil, rapeseed oil, nut oils and soybean oil serve as preferred sources of cc-linolenic acid, algae, algal products (for example oils) or products or oils from other marine microorganisms, and also fish oil, serve as preferred sources of DHA and EPA. Thistle oil, sunflower oil, soybean oil and maize germ oil are preferred sources of linoleic acid, evening primrose oil and borage oil are preferred sources of γ-linolenic acid (gamma-linolenic acid), and olive oil and rapeseed oil of oleic acid.

As sources of DHA, in particular, preference is given to oil from microorganisms, in particular from the group of the Thraustochytriales and Dinophyta.

A further embodiment of the present invention relates to the production of the inventive dietetic preparation by mixing desired amounts of dietary fiber and n3 fatty acids and also if appropriate other components. This mixture can thereupon be spray dried, freed from solvent, agglomerated and/or instantized. Furthermore, use can be made of all current food engineering or pharmaceutical production methods such as pressing, kneading or dragee-coating. The n3 fatty acids can be added to the mixture in pure form or encapsulated or microencapsulated, use being able to be made for encapsulation or microencapsulation of all methods familiar to those skilled in the art, such as coacervation, spray drying or fluidized-bed drying. Incorporation into liposomes or micelles is also possible.

In addition, the n3 fatty acids can be added to the mixture in a form which permits continuous (retarding) release of the fatty acids in the body. Suitable methods for producing these “slow release” formulations are, for example, coating methods or the use of suitable encapsulating matrices in the (micro)encapsulation.

In addition, the inventive dietary fiber, or the further constituents (for example carbohydrates, protein, fat) can itself or themselves be used as support or matrix for the n3 fatty acids.

The inventive combinations can also be administered in the form of two or more different administration forms.

A further preferred embodiment of the present invention relates to the use of a dietetic preparation as described above for producing a medical food composition for weight control or weight reduction.

In this context it is conceivable to use the dietetic preparation in combination with the active compound orlistat or else with chitosan, to name only two examples, to produce a medical food composition for weight control or weight reduction.

A further very preferred embodiment of the present invention relates to the use of a dietetic preparation as described above for producing a food for a weight-controlling or weight-reducing diet.

In this case, suitable applications are current food applications such as bakery products, e.g. bread, rolls, cereals, snack or fruit bars or drinks powders.

Furthermore, the direct addition of the inventive dietetic preparation to self-prepared foods (for example in the form of (possibly soluble) tablets, dragees, hard or soft capsules or sachets, granules, powders) is also possible.

A further very preferred embodiment of the present invention relates to use of a dietetic preparation as described above for producing a food supplement in a weight-controlling or weight-reducing diet, or for nutritional support in diets.

Articles which come into consideration for this are, in particular, snack or fruit bars, drinks or drinks powders, pulverulent or tablet-form preparations for dissolution, or else tablets (e.g.: chewing tablets), dragees, hard or soft capsules, sachets or granules.

A preferred embodiment of the present invention therefore relates to dietetic foods, or food additives, for weight control or weight reduction comprising at least one n3 fatty acid and at least one dietary fiber and also a ratio as already described above of carbohydrates, protein and fat.

The inventive food is suitable, in the context of a dietary plan for weight reduction, or weight control, for compensating for a diet-related deficit of n3 fatty acids, and also making a positive contribution to normalizing the triglyceride and cholesterol values and thus exerting a pronounced beneficial effect on cardiovascular health.

In addition, the inventive active compound combination can compensate, in particular, for a depletion of the body in essential n3 fatty acids which experience has shown can result after administration of dietary fibers and, in particular, as an undesired side reaction in a drug treatment of high cholesterol values with statins. The treatment with statins is indicated for overweight and adiposity patients, and also for diabetics and heart patients. A depletion of essential n3 fatty acids can also occur as a consequence of a targeted weight reduction frequently recommended for these patients, in particular by reducing the fat proportion in the diet.

In addition, the inventive active compound combination can comprise drugs or dietetic agents for reducing the fat uptake in the intestine, e.g. the active compound orlistat (Xenical, Roche) or else chitosan products. However, it is necessary to ensure here that depletion of the body in essential fatty acids is compensated for, which depletion can occur as an undesired side reaction of the intake of orlistat or chitosan products. In the case of a mixture comprising said active compounds orlistat (Xenical, from Roche) or chitosan products or with simultaneous oral supply of these substances, the preferred daily dose of total omega-3 fatty acids is about 70 mg-3 g. In particular preference for this case is given to a concentration of DHA at 100 mg-2 g, particularly preferably 120 mg-800 mg.

The inventive dietetic foods comprise the n3 fatty acids and dietary fibers in amounts which are suitable, in the case of single or else repeated daily intake for achieving the desired daily dose or to compensate for a decreased physiological utilizability of fatty acids, e.g. with simultaneous increase in dietary fibers. The dosage of the individual components can also be adjusted individually to the respective patient and his or her nutritional or medical requirements, or cover the typical requirements in one or more simple mixtures. Depending on the desired result, different mixtures of the components can be used.

In addition, the n3 fatty acids are also present in the inventive dietetic foods in concentrations which beneficially affect health (or correspond to the recommendations). The daily dose of n3 fatty acids can be in the range from 50 mg to 10 g, customarily from 100 mg to 7 g, and preferably from 200 mg to 5 g.

Particularly preferably, the inventive dietetic foods comprise the fatty acids in a concentration resulting in the following daily doses:
n3 fatty acids: 0.5-5 g of α-linolenic acid; in a particular embodiment, in addition 50 mg-2 g of long-chain n3 fatty acids (DHA and/or EPA)
n6 fatty acids: 2-10 g of linofeic acid, in a particular embodiment, in addition 100-500 mg γ-linolenic acid,

the ratio of n3 to n6 fatty acids preferably being between 1:1 and 1: 10, particularly preferably between 1:1 and 1:5.

In a particularly preferred embodiment, the inventive dietetic food also comprises at least 2 g of the monounsaturated oleic acid.

A particularly preferred example of an inventive dietetic food administers daily approximately 1500-2000 kcal, with the following composition being selected for the fatty acids: n3 fatty acids: ALA, DHA, EPA: in total 1-10 g, preferably 3-8 g, ALA making up 2-6 g thereof and DHA 100-300 mg, preferably approximately 200 mg; n6 fatty acids: LA 4-20 g, preferably no more than 10 g. The ratio of LA to ALA/DHA/EPA is preferably to be between 1:1 and 10:1.

The inventive dietetic foods can be taken at a certain daily timepoint, or distributed over the day, the ratios of dietary fibers and n3 fatty acids in the case of intake of smaller doses corresponding to the abovementioned ratios.

The inventive compositions may be administered in combination, or else separately in the form of a dietetic food or food supplement comprising the dietary fiber and the n3 fatty acids.

The inventive compositions can of course comprise all other additives appearing useful for the purpose to those skilled in the art. Those which may be mentioned by way of example are components (additives) for improving the dissolution, such as soluble carriers or tablet disintegrants, e.g. starch, cellulose, bentonite, pectin or peroxides and carbonates in combination with organic acids, antioxidants and preservatives or generally colorants, flavorings, sweeteners such as sucrose, glucose, fructose and other carbohydrates, sugar alcohols, for example sorbitol, xylitol, maltitol and isomalt, or high-intensity sweeteners, e.g. acesulfame-K, cyclamate, saccharin, sucralose or aspartame and in particular aroma substances (for example vanillin) to improve acceptance. Furthermore, the compositions can comprise customary additives such as solvents, fillers, supports such as methyl cellulose, inter alia.

### EXAMPLES

The following examples are quoted to explain preferred embodiments of the invention. Those skilled in the art should understand that the preparations and foods disclosed in the following examples have been characterized by the inventors as functioning well when the present invention is carried out. They can therefore be considered as preferred embodiments. However, those skilled in the art should understand in the light of the present disclosure that many changes in the specific disclosed embodiments are possible, the same or at least a similar result still being achieved, without departing from the context of the invention.

### Example 1

Pulverulent Preparation for Food Supplementation (for One Portion Size)

| | | |
|---|---|---|
| Actistar ™ (Cerestar) | 3 | g |
| DHA-rich algal oil (DHA content 43%) | 120 | mg |
| Xanthan (stabilizer) | 150 | mg |
| Vanillin | 15 | mg |

Suspend the preparation by stirring in 150 ml of tepid low-fat milk and drink.

### Example 2

Drink Powder as Meal Substitute, Enriched with Nutrients and Vitamins.

Portion size 35 g. Stir into 250 ml of water or low-fat milk.

Ingredients: sugar, thickeners, microcrystals, fructooligosaccharides (3 g), vegetable oil (e.g. mixture of olive, linseed, sunflower, thistle, evening primrose, rapeseed, soybean, palm oils inter alia), cocoa powder, inulin, DHA-rich algal oil (43% DHA, 150 mg), milk protein, chocolate powder, vanillin, maltodextrin, skimmed-milk powder.

Minerals and vitamins per portion (35 g):

potassium citrate 210 mg, sodium citrate 110 mg, magnesium oxide 38.5 mg, iron sulfate 13.76 mg, zinc oxide 5.53 mg, copper gluconate 2.50 mg, manganese sulfate 0.82 mg, potassium iodide 0.07 mg, sodium selenite 0.03 mg; vitamin C 42.51 mg, vitamin E 17.33 mg, niacin 6.38 mg, pantothenate 1.72 mg, vitamin B6 0.86 mg, vitamin A 0.70 mg, vitamin B1 0.67 mg, vitamin B2 0.13 mg, folic acid 0.07 mg, biotin 0.07 mg, vitamin D 0.002 mg. Antioxidants: sodium ascorbate, tocopherol.

Color, thickener: carboxymethylcellulose, xanthan

### Example 3

Finished Drink, Vanilla (Portion Size 250 ml)

Components:

skimmed milk; water; sugar; milk protein; fructooligosaccharides; thickener, linseed oil, sunflower oil, soybean oil, DHA-rich algal oil, emulsifier: E 471; potassium chloride, magnesium hydrogen carbonate, sodium chloride, zinc gluconate, iron phosphate, calcium pantothenate, copper gluconate, manganese sulfate, potassium iodide, sodium selenite, vitamin C, B-carotene, vitamin E, niacin, vitamin B6, vitamin B1, vitamin D, vitamin B2, vitamin A, folic acid, biotin.

### Example 4

Bar (30 g)

3 g of Novellose 330™ (National Starch) and 100 mg of DHA-rich algal oil (43% DHA) incorporated into a bar base substrate consisting of:

milk protein, soy protein, sucrose, hazelnut crocant, sorbitol syrup, lactose product, sultanas, starch syrup, vegetable fat (linseed oil, rapeseed oil, sunflower oil, evening primrose oil) part hardened, almonds, roller-dried cream powder, cornflakes, invert sugar syrup, soy, wafers, milk caramel powder, vitamin C, flavoring, emulsifier: lecithin, niacin, vitamin E, pantothenic acid, vitamins B2, B6, B1, folic acid, biotin, vitamin B12.

1. A Dietetic preparation comprising
a) at least one n3 fatty acid,
b) at least one dietary fiber selected from the list consisting of: resistant starch, fructooligosaccharides, oligofructoses and/or inulin,
c) if appropriate carbohydrates,
d) if appropriate protein, and also
e) if appropriate fat.

2. The dietetic preparation as claimed in claim 1, comprising as dietary fiber a mixture of resistant starch and inulin in the ratio 1:10 to 10:1.

3. The dietetic preparation as claimed in claim 1, the total energy content of the composition where the corresponding component is present being 40-75% covered, preferably 55-60%, by component c), 5-35% covered, preferably 10-15%, by component d) and 15-35% covered, preferably 25-30%, by component e).

4. The dietetic preparation as claimed in claim 1, further comprising at least one n6 fatty acid, the ratio of n3 fatty acids and n6 fatty acids ranging from 1:1 to 1: 15, and the at least one n6 fatty acid preferably being selected from the group consisting of linoleic acid, γ-linolenic acid, arachidonic acid and/or n6-DPA.

5. The dietetic preparation as claimed in claim 1, wherein at least one n3 fatty acid is selected from the group consisting of docosahexaenoic acid, eicosapentaenoic acid and/or α-linolenic acid.

6. The dietetic preparation as claimed in claim 1, comprising a plurality of n3 fatty acids and/or a plurality of dietary fibers.

7. The dietetic preparation as claimed in claim 1. further comprising vitamins, minerals, trace elements, further essential fatty acids and/or secondary plant materials.

8. The dietetic preparation as claimed in claim 1, wherein saturated fatty acids contribute at most to 10% of the energy which this preparation provides to an organism.

9. The dietetic preparation as claimed in claim 1, comprising at least one monounsaturated fatty acid.

10. The dietetic preparation as claimed in claim 1, comprising linoleic acid, α-linolenic acid, docosahexaenoic acid and/or eicosapentaenoic acid, linoleic acid contributing 1 to 10% of the energy which this food provides to the organism, α-linolenic acid 0.2-2%, docosahexaenoic acid and/or eicosapentaenoic acid together making up 0.1-1%, DHA making up at least 0.1%.

11. The dietetic preparation as claimed in claim 1, comprising one of the following oils: algal oil, borage oil, thistle oil, fish oil, hemp oil, linseed oil, maize germ oil, evening primrose oil, olive oil, rapeseed oil, soybean oil, palm oil, walnut oil, coconut oil and/or sunflower oil.

12. A dietetic preparation comprising
a) at least one n3 fatty acid and also if appropriate at least one n6 fatty acid, the ratio of n3 fatty acids to n6 fatty acids ranging from 1:1 to 1:10, and the at least one n6 fatty acid preferably being selected from the group consisting of linoleic acid, γ-linolenic acid and/or arachidonic acid, and the at least one n3 fatty acid from the group consisting of docosahexaenoic acid, eicosapentaenoic acid and/or a-linolenic acid,
b) if appropriate carbohydrates,
c) if appropriate protein,
d) if appropriate fat,
e) if appropriate a dietary fiber and
e) at least one active compound which reduces the uptake of fat in the intestine, the total energy content of the composition being 40-75% covered, preferably 55-60%, by component b), 5-35% covered, preferably 10-15%, by component c) and 15-35% covered, preferably 25-30%, by component d).

13. The dietetic preparation as claimed in claim 12, the active compound being orlistat and/or chitosan.

14. The use of a dietetic preparation as claimed in claim 12 for producing a medical food composition for weight control or weight reduction.

15. The use of a dietetic preparation as claimed as claimed in claim 1 for producing a food for a weight-controlling or weight-reducing diet.

16. The use of a dietetic preparation as claimed in claim 1 for producing a snack product for a weight-controlling or weight-reducing diet.

17. The use of a dietetic preparation as claimed in claim 1 for producing a food supplement in a weight-controlling or weight-reducing diet.

18. The use of a dietetic preparation as claimed in claim 1 for producing a food supplement for nutritional support in diets.

19. The medical food composition as claimed in claim 14 for weight control or weight reduction.

20. A food for the weight-controlling or weight-reducing diet as claimed in claim 15.

21. A food supplement in a weight-controlling or weight-reducing diet as claimed in claim 17.
